(19) United States
(12) Patent Application Publication
(10) Pub. No.: US 2007/0149578 A1
(43) Pub. Date: Jun. 28, 2007

(54) Combination Therapy

(75) Inventors: Jan Buch, Greenwich, CT (US); Robert Andrew Donald Scott, Riverside, CT (US)

Correspondence Address: CONNOLLY BOVE LODGE & HUTZ, LLP, P O BOX 2207, WILMINGTON, DE, 19899, US

(73) Assignee: Pfizer, Inc., Morris Plains, NJ (US)

(21) Appl. No.: 11/549,697

(22) Filed: Oct. 16, 2006

Related U.S. Application Data
(63) Continuation of application No. 10/879,937, filed on Jun. 29, 2004, now abandoned.
Application No. 10/879,937 is a continuation of application No. 10/336,463, filed on Jan. 2, 2003, now abandoned.
Application No. 10/336,463 is a continuation of application No. 09/975,765, filed on Oct. 10, 2001, now abandoned.
Application No. 09/975,765 is a continuation of application No. 09/513,889, filed on Feb. 25, 2000, now abandoned.

(51) Int. Cl. A61K 31/455; A61K 31/401; A61K 31/366; A61K 31/22

(52) U.S. Cl. 514/355; 514/423; 514/460; 514/548

(57) ABSTRACT

This invention relates to pharmaceutical combinations of amlodipine or a pharmaceutically acceptable acid addition salt thereof and statins or pharmaceutically acceptable salts thereof, kits containing such combinations and methods of using such combinations to treat subjects suffering from angina pectoris, atherosclerosis, combined hypertension and hyperlipidemia and to treat subjects presenting with symptoms of cardiac risk, including humans. This invention also relates to additive and synergistic combinations of amlodipine or a pharmaceutically acceptable acid addition salt thereof and statins or pharmaceutically acceptable salts thereof whereby those additive and synergistic combinations are useful in treating subjects suffering from angina pectoris, atherosclerosis, combined hypertension and hyperlipidemia and those subjects presenting with symptoms of cardiac risk, including humans.

### COMBINATION THERAPY

This invention relates to pharmaceutical combinations of amiodipine or pharmaceutically acceptable acid addition salts thereof and statins and pharmaceutically acceptable salts thereof, kits containing such combinations and methods of using such combinations to treat subjects suffering from angina pectoris, atherosclerosis, combined hypertension and hyperlipidemia and to treat subjects presenting with symptoms of cardiac risk, including humans. This invention also relates to additive and synergistic combinations of amlodipine or a pharmaceutically acceptable acid addition salt and statins or pharmaceutically acceptable salts thereof whereby those additive and synergistic combinations are useful in treating subjects suffering from angina pectoris, atherosclerosis, combined hypertension and hyperlipidemia and those subjects presenting with symptoms or signs of cardiac risk, including humans.

### BACKGROUND OF THE INVENTION

The conversion of 3-hydroxy-3-methylglutarylcoenzyme A (HMG-CoA) to mevalonate is an early and rate-limiting step in the cholesterol biosynthetic pathway. This step is catalyzed by the enzyme HMG-CoA reductase. Statins inhibit HMG-CoA reductase from catalyzing this conversion. As such, statins are collectively potent lipid lowering agents. Statins include such compounds as simvastatin, disclosed in U.S. Pat. No. 4,444,784, which is incorporated herein by reference; pravastatin, disclosed in U.S. Pat. No. 4,346,227 which is incorporated herein by reference; cerivastatin, disclosed in U.S. Pat. No. 5,502,199, which is incorporated herein by reference; mevastatin, disclosed in U.S. Pat. No. 3,983,140, which is incorporated herein by reference; velostatin, disclosed in U.S. Pat. No. 4,448,784 and U.S. Pat. No. 4,450,171, both of which are incorporated herein by reference; fluvastatin, disclosed in U.S. Pat. No. 4,739,073, which is incorporated herein by reference; compactin, disclosed in U.S. Pat. No. 4,804,770, which is incorporated herein by reference; lovastatin, disclosed in U.S. Pat. No. 4,231,938, which is incorporated herein by reference; dalvastatin, disclosed in European Patent Application Publication No. 738510 A2; fluindostatin, disclosed in European Patent Application Publication No. 363934 A1; atorvastatin, disclosed in U.S. Pat. No. 4,681,893, which is incorporated herein by reference; atorvastatin calcium, disclosed in U.S. Pat. No. 5,273,995, which is incorporated herein by reference; and dihydrocompactin, disclosed in U.S. Pat. No. 4,450,171, which is incorporated herein by reference.

Amlodipine and related dihydropyridine compounds are disclosed in U.S. Pat. No. 4,572,909, which is incorporated herein by reference, as potent anti-ischemic and antihypertensive agents. U.S. Pat. No. 4,879,303, which is incorporated herein by reference, discloses amlodipine benzenesulfonate salt (also termed amlodipine besylate). Amlodipine and amlodipine besylate are potent and long lasting calcium channel blockers. As such, amlodipine, amlodipine besylate and other pharmaceutically acceptable acid addition salts of amlodipine have utility as antihypertensive agents and as antiischemic agents. Amlodipine and its pharmaceutically acceptable acid addition salts are also disclosed in U.S. Pat. No. 5,155,120 as having utility in the treatment of congestive heart failure. Amlodipine besylate is currently sold as Norvasc®. Amlodipine has the formula

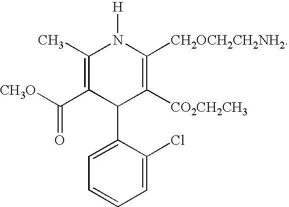

Atherosclerosis is a condition characterized by irregularly distributed lipid deposits in the intima of arteries, including coronary, carotid and peripheral arteries. Atherosclerotic coronary heart disease (hereinafter termed “CHD”) accounts for 53% of all deaths attributable to a cardiovascular event. CHD accounts for nearly one-half (about $50-60 billion) of the total U.S. cardiovascular healthcare expenditures and about 6% of the overall national medical bill each year. Despite attempts to modify secondary risk factors such as, inter alia, smoking, obesity and lack of exercise, and treatment of dyslipidemia with dietary modification and drug therapy, CHD remains the most common cause of death in the United States.

High levels of blood cholesterol and blood lipids are conditions involved in the onset of atherosclerosis. It is well known that inhibitors of 3hydroxy-3-methylglutaryl-coenzyme A reductase (HMG-CoA reductase) are effective in lowering the level of blood plasma cholesterol, especially low density lipoprotein cholesterol (LDL-C), in man (Brown and Goldstein, New England Journal of Medicine, 1981, 305, No. 9,- 515-517). It has now been established that lowering LDL-C levels affords protection from coronary heart disease (see, e.g., The Scandinavian Simvastatin Survival Study Group: Randomised trial of cholesterol lowering in 4444 patients with coronary heart disease: the Scandinavian Simvastatin Survival Study (4S), Lancet, 1994, 344, 1383-89; and Shepherd, J. et al., Prevention of coronary heart disease with pravastatin in men with hypercholesterolemia, New England Journal of Medicine, 1995, 333, 1301-07).

Angina pectoris is a severe constricting pain in the chest often radiating from the precordium to the left shoulder and down the left arm. Often angina pectors is due to ischemia of the heart and is usually caused by coronary disease.

Currently the treatment of symptomatic angina pectoris varies significanty from country to country. In the U.S., patients who present with symptomatic, stable angina pectoris are frequently treated with surgical procedures or PTCA. Patients who undergo PTCA or other surgical procedures designed to treat angina pectoris frequently experience complications such as restenosis. This restenosis may be manifested either as a short term proliferative response to angioplasty-induced trauma or as long term progression of the atherosclerotic process in both graft vessels and angioplastied segments.

The symptomatic management of angina pectoris involves the use of a number of drugs, frequently as a combination of two or more of the following classes: beta blockers, nitrates and calcium channel blockers. Most, if not all, of these patients require therapy with a lipid lowering agent as well. The National Cholesterol Education Program (NCEP) recognizes patients with existing coronary artery disease as a special class requiring aggressive management of raised LDL-C.

Amlodipine helps to prevent myocardial ischemia in patients with exertional angina pectoris by reducing Total Peripheral Resistance, or afterload, which reduces the rate pressure product and thus myocardial oxygen demand at any particular level of exercise. In patients with vasospastic angina pectoris, amlodipine has been demonstrated to block constriction and thus restore myocardial oxygen supply. Further, amlodipine has been shown to increase myocardial oxygen supply by dilating the coronary arteries.

Hypertension frequently coexists with hyperlipidemia and both are considered to be major risk factors for developing cardiac disease ultimately resulting in adverse cardiac events. This clustering of risk factors is potentially due to a common mechanism. Further, patient compliance with the management of hypertension is generally better than patient compliance with hyperlipidemia. It would therefore be advantageous for patients to have a single therapy which treats both of these conditions.

Coronary heart disease is a multifactorial disease in which the incidence and severity are affected by the lipid profile, the presence of diabetes and the sex of the subject. Incidence is also affected by smoking and left venticular hypertrophy which is secondary to hypertension. To meaningfully reduce the risk of coronary heart disease, it is important to manage the entire risk spectrum. For example, hypertension intervention trials have failed to demonstrate full normalization in cardiovascular mortality due to coronary heart disease. Treatment with cholesterol synthesis inhibitors in patients with and without coronary artery disease reduces the risk of cardiovascular morbidity and mortality.

The Framingham Heart Study, an ongoing prospective study of adult men and women, has shown that certain risk factors can be used to predict the development of coronary heart disease. (see Wilson et al., Am. J. Cardiol. 1987, 59(14):91G-94G). These factors include age, gender, total cholesterol level, high density lipoprotein (HDL) level, systolic blood pressure, cigarette smoking, glucose intolerance and cardiac enlargement (left ventricular hypertrophy on electrocardiogram, echocardiogram or enlarged heart on chest X-ray). Calculators and computers can easily be programmed using a mulfivariate logistic function that allows calculation of the conditional probability of cardiovascular events. These determinations, based on experience with 5,209 men and women participating in the Framingham study, estimate coronary artery disease risk over variable periods of follow-up. Modeled incidence rates range from less than 1% to greater than 80% over an arbitrarily selected six year interval. However, these rates are typically less than 10% and rarely exceed 45% in men and 25% in women.

Kramsch et al., Journal of Human Hypertension (1995) (Suppl. 1), 5359 discloses the use of calcium channel blockers, including amlodipine, to treat atherosclerosis. That reference further suggests that atherosclerosis can be treated with a combination of amlodipine and a lipid lowering agent Human trials have shown that calcium channel blockers have beneficial effects in the treatment of early atherosclerotic lesions. (see, e.g., Lichtien, P. R. et al., Retardation of angiographic progression o coronary artery disease by nifedipine, Lancet, 1990, 335, 1109-913; and Waters, D. et al., A controlled clinical trial to assess the effect of a calcium channel blocker on the progression of coronary atherosclerosis, Circulation, 1990, 82, 194053.) U.S. Pat. No. 4,681,893 discloses that certain statins including atorvastatin, are hypolipidemic agents and as such are useful in treating atherosclerosis. Jukema et al., Circulation, 1995 (Suppl. 1), 1-197 disclose that there is evidence that calcium channel blockers act synergistically in combination with lipid lowering agents (e.g., HMG-CoA reductase inhibitors), specifically pravastatin. Orekhov et al., Cardiovascular Drugs and Therapy, 1997, 11, 350 disclose the use of amlodipine in combination with lovastatin for the treatment of atherosclerosis.

### SUMMARY OF THE INVENTION

This invention is directed to a pharmaceutical composition, hereinafter termed “Composition A”, comprising an amount of amlodipine or a pharmaceutically acceptable acid addition salt thereof, an amount of a statin or a pharmaceutically acceptable salt thereof, and a pharmaceutically acceptable carrier, provided that said statin is not atorvastatin or a pharmaceutically acceptable salt thereof.

This invention is particularly directed to a pharmaceutical composition, hereinafter termed “Composition AA”, of Composition A wherein said statin is simvastatin, pravastatin, rivastatin, mevastatin, fluindostatin, velostatin, fluvastatin, dalvastatin, dihydrocompactin, compactin or lovastatin; or a pharmaceutically acceptable salt of simvastatin, pravastatin, rivastatin, mevastatin, fluindostatin, velostatin, fluvastatin, dalvastatin, dihydrocompactin, compactin or lovastatin.

This invention is particularly directed to a pharmaceutical composition, hereinafter termed “Composition AB”, of Composition AA wherein said statin is simvastatin, pravastatin, rivastatin, mevastatin, fluindostatin, velostatin, fluvastatin, dalvastatin, dihydrocompactin or compacting or a pharmaceutically acceptable salt of simvastatin, pravastatin, rivastatin, mevastatin, fluindostatin, velostatin, fluvastatin, dalvastatin, dihydrocompactin or compactin.

This invention is more particularly directed to a pharmaceutical composition, hereinafter termed “Composition AB”, of Composition AA wherein said statin is simvastatin, pravastatins mevastatin or pharmaceutically acceptable salts thereof.

This invention is still more particularly directed to a pharmaceutical composition of Composition AB comprising amlodipine besylate.

This invention is also directed to a first pharmaceutical composition, hereinafter termed “Composition B”, for use with a second pharmaceutical composition for achieving a antihypertensive effect and a hypolipidemic effect in a mammal suffering from hypertension and hyperlipidemia, which effects are greater than-the sum of the antihypertensive and hypolipidemic effects achieved by administering said first and second pharmaceutical compositions separately and which second pharmaceutical composition comprises an amount of amlodipine or a pharmaceutically acceptable acid addition salt thereof and a pharmaceutically acceptable carrier or diluent, said first pharmaceutical composition comprising an amount of a statin or a pharmaceutically acceptable salt thereof and a pharmaceutically acceptable carrier or diluent; provided that said statin is not atorvastatin or a pharmaceutically acceptable salt thereof.

This invention is particularly directed to a composition, hereinafter termed “Composition BA”, of Composition B wherein said statin is simvastatin, pravastatin, rivastatin, mevastatin, fluindostatin, velostatin, fluvastatin, dalvastatin, dihydrocompactin, compactin or lovastatin; or a pharmaceutically acceptable salt of sirhvastatin, pravastatin, rivastatin, mevastatin, fluindostatin, velostatn, fluvastatin, dalvastatin, dihydrocompactin, compactin or lovastatin.

This invention is particularly directed to a composition, hereinafter termed “Composition BB”, of Composition BA wherein said statin is simvastatin, pravastatin, rivastatin, mevastatin, fluindostatin, velostatin, fluvastatin, dalvastatin, dihydrocompactin or compactin; or a pharmaceutically acceptable salt of simvastatin, pravastatin, rivastatin, mevastatin, fluindostatin, velostatin, fluvastatin, dalvastatin, dihydrocompactin or compactin.

This invention is more particularly directed to a composition of Composition BA wherein said second composition comprises amlodipine besylate.

This invention is also directed to a first pharmaceutical composition, hereinafter termed “C”, for use with a second pharmaceutical composition for achieving a antihypertensive effect and a hypolipidemic effect in a mammal suffering from hypertension and hyperlipidemia, which effects are greater than the sum of the antihypertensive and hypolipidemic effects achieved by administering said first and second pharmaceutical compositions separately and which second pharmaceutical composition comprises an amount of a statin or a pharmaceutically acceptable salt thereof and a pharmaceutically acceptable carrier or diluent, said first pharmaceutical composition comprising an amount of amlodipine or a pharmaceutically acceptable acid addition salt thereof and a pharmaceutically acceptable carrier or diluent, provided that said statin is not atorvastatin or a pharmaceutically acceptable salt thereof.

This invention is particularly directed to a composition, hereinafter termed “Composition CA”, of Composition C wherein said statin is simvastatin, pravastatin, rivastatin, mevastatin, fluindostatin, velostatin, fluvastatin, dalvastatin, dihydrocompactin, compactin or lovastatin; or a pharmaceutically acceptable salt of simvastatin, pravastatin, rivastatin, mevastatin, fluindostatin, velostatin, fluvastatin, dalvastatin, dihydrocompactin, compactin or lovastatin.

This invention is particularly directed to a composition, hereinafter termed “Composition CB”, of Composition CA wherein said statin is simvastatin, pravastatin, rivastatin, mevastatin, fluindostatin, velostatin, fluvastatin, dalvastatin, dihydrocompactin or compactin; or a pharmaceutically acceptable salt of simvastatin, pravastatin, rivastatin, mevastatin, fluindostatin, velostatin, fluvastatin, dalvastatin, dihydrocompactin or compactin.

This invention is still more particularly directed to a composition of Composition CA comprising amlodipine besylate.

This invention is also directed to a first pharmaceutical composition, hereinafter termed “Composition D”, for use with a second pharmaceutical composition for achieving a antihypertensive effect and a hypolipidemic effect in a mammal suffering from hypertension and hyperlipidemia, which effects are greater than the antihypertensive and hypolipidemic effects achieved by administering said first or second pharmaceutical compositions separately and which second pharmaceutical composition comprises an amount of a statin or a pharmaceutically acceptable salt thereof and a pharmaceutically acceptable carrier or diluent, said first pharmaceutical composition comprising an amount of amlodipine or a pharmaceutically acceptable acid addition salt thereof and a pharmaceutically acceptable carrier or diluent, provided that said statin is not atorvastatin or a pharmaceutically acceptable salt thereof.

This invention is still more particularly directed to a composition of Composition D comprising amlodipine besylate.

This invention is also directed to a first pharmaceutical composition, hereinafter termed “Composition E”, for use with a second pharmaceutical composition for achieving a antihypertensive effect and a hypolipidemic effect in a mammal suffering from hypertension and hyperlipidemia, which effects are greater than the antihypertensive and hypolipidemic effects achieved by administering said first or second pharmaceutical compositions separately and which second pharmaceutical composition comprises an amount of amlodipine or a pharmaceutically acceptable acid addition salt thereof and a pharmaceutically acceptable carrier or diluent, said first pharmaceutical composition comprising an amount of a statin or a pharmaceutically acceptable salt thereof and a pharmaceutically acceptable carrier or diluent; provided that said stain is not atorvastatin or a pharmaceutically acceptable salt thereof.

This invention is particularly directed to a composition, hereinafter termed “Composition EA”, of Composition E wherein said statin is simvastatin, pravastatin, rivastatin, mevastatin, fluindostatin, velostatin, fluvastatin, dalvastatin, dihydrocompactin, compactin or lovastatin; or a pharmaceutically acceptable salt of simvastatin, pravastatin, rivastatin, mevastatin, fluindostatin, velostatin, fluvastatin, dalvastatin, dihydrocompactin, compactin or lovastatin.

This invention is particularly directed to a composition of Composition EA wherein said statin is simvastatin, pravastatin, rivastatin, mevastatin, fluindostatin, velostatin, fluvastatin, dalvastatin, dihydrocompactin or compactin; or a pharmaceutically acceptable salt of simvastatin, pravastatin rivastatin, mevastatin, fluindostatin, velostatin, fluvastatin, dalvastatin, dihydrocompactin or compactin.

This invention is further directed to a first pharmaceutical composition, hereinafter termed “Composition F”, for use with a second pharmaceutical composition for achieving an antianginal effect in a mammal suffering from angina pectoris, which effect is greater than the sum of the antiangina effects achieved by administering said first and second pharmaceutical compositions separately and which second pharmaceutical composition comprises an amount of a statin or a pharmaceutically acceptable salt thereof and a pharmaceutically acceptable carrier or diluent, said first pharmaceutical composition comprising an amount of amlodipine or a pharmaceutically acceptable acid addition salt thereof and a pharmaceutically acceptable carrier or diluent; provided that said statin is not atorvastatin or a pharmaceutically acceptable salt thereof.

This invention is particularly directed to a composition, hereinafter termed “Composition FA”, of Composition F wherein said statin is simvastatin, pravastatin, rivastatin, mevastatin, fluindostatin, velostatin, fluvastatin, dalvastatin, dihydrocompactin, compactin or lovastatin; or a pharmaceutically acceptable salt of simvastatin, pravastatin, rivastatin, mevastatin, fluindostatin, velostatin, fluvastatin, dalvastatin, dihydrocompactin, compactin or lovastatin.

This invention is particularly directed to a composition, hereinafter termed “Composition FB”, of Composition FA wherein said statin is simvastatin, pravastatin, rivastatin, mevastatin, fluindostatin, velostatin, fluvastatin, dalvastatin, dihydrocompactin or compactin; or a pharmaceutically acceptable salt of simvastatin, pravastatin, rivastatin, mevastatin, fluindostatin, velostatin, fluvastatin, dalvastatin, dihydrocompactn or compactin.

This invention is more particularly directed to a composition of Composition FA comprising amlodipine besylate.

This invention is also directed to a first pharmaceutical composition$ hereinafter termed “Composition G”, for use with a second pharmaceutical composition for achieving an antianginal effect in a mammal suffering from angina pectoris, which effect is greater than the sum of the antiangina effects achieved by administering said first and second pharmaceutical compositions separately and which second pharmaceutical composition comprises an amount of amlodipine or a pharmaceutically acceptable acid addition salt thereof and a pharmaceutically acceptable carrier or diluent, said first pharmaceutical composition comprising an amount of a statin or a pharmaceutically acceptable salt thereof and a pharmaceutically acceptable carrier or diluent; provided that said statin is not atorvastatin of a pharmaceutically acceptable salt thereof.

This invention is particularly directed to a composition, hereinafter termed “Composition GA”, of Composition G wherein said statin is simvastatin, pravastatin, rivastatin, mevastatin, fluindostatin, velostatin, fluvastatin, dalvastatin, dihydrocompactin, compactin or lovastatin; or a pharmaceutically acceptable salt of simvastatin, pravastatin, rivastatin, mevastatin, fluindostatin, velostatin, fluvastatin, dalvastatin, dihydrocompactin, compactin or lovastatin.

This invention is particularly directed to a composition, hereinafter termed “Composition GB”, of Composition GA wherein said statin is simvastatin, pravastatin, rivastatin, mevastatin, fluindostatin, velostatin, fluvastatin, dalvastatin, dihydrocompactin or compactin; or a pharmaceutically acceptable salt of simvastatin, pravastatin, rivastatin, mevastatin, fluindostatin, velostatin, fluvastatin, dalvastatin, dihydrocompactin or compactin.

This invention is more particularly directed to a composition of Composition GA wherein said second pharmaceutical composition comprises amlodipine besylate.

This invention is also directed to a first pharmaceutical composition, hereinafter termed “Composition H”, for use with a second pharmaceutical composition for achieving an antianginal effect in a mammal suffering from angina pectoris, which effect is greater than the antianginal effects achieved by administering said first or second pharmaceutical compositions separately and which second pharmaceutical composition comprises an amount of a statin or a pharmaceutically acceptable salt thereof and a pharmaceutically acceptable carrier or diluent, said first pharmaceutical composition comprising an amount of amlodipine or a pharmaceutically acceptable acid addition salt thereof and a pharmaceutically acceptable carrier or diluent; provided that said statin is not atorvastatin or a pharmaceutically acceptable salt thereof.

This invention is still more particularly directed to a pharmaceutical composition of Composition H comprising amlodipine besylate.

This invention is also directed to a first pharmaceutical composition, hereinafter termed “Composition J”, for use with a second pharmaceutical composition for achieving an antianginal effect in a mammal suffering from angina pectoris, which effect is greater than the antianginal effects achieved by administering said first or second pharmaceutical compositions separately and which second pharmaceutical composition comprises an amount of amlodipine or a pharmaceutically acceptable acid addition salt thereof and a pharmaceutically acceptable carrier or diluent said first pharmaceutical composition comprising an amount of a statin or a pharmaceutically acceptable salt thereof and a pharmaceutically acceptable carrier or diluent; provided that said statin is not atorvastatin or a pharmaceutically-acceptable salt thereof.

This invention is particularly directed to a composition, hereinafter termed “Composition JA”, of Composition J wherein said statin is simvastatin, pravastatin, rivastatin, mevastatin, fluindostatin, velostatin, fluvastatin, dalvastatin, dihydrocompactn, compactin or lovastatin; or a pharmaceutically acceptable salt of simvastatin, pravastatin, rivastatin, mevastatin, fluindostatin, velostatin, fluvastatin dalvastatin, dihydrocompactin, compactin or lovastatin.

This invention is particularly directed to a composition, hereinafter termed “Composition JB”, of Composition JA wherein said statin is simvastatin, pravastatin, rivastatin, mevastatin, fluindostatin, velostatin, fluvastatin, dalvastatin, dihydrocompactin or compactin; or a pharmaceutically acceptable salt of simvastatin, pravastatin, rivastatin, mevastatin, fluindostatin, velostatin, fluvastatin, dalvastatin, dihydrocompactin or compactin.

This invention is also directed to a first pharmaceutical composition, hereinafter termed “Composition K”, for use with a second pharmaceutical composition for achieving an antiatherosclerotic effect in a mammal, which effect is greater than the sum of the antiatherosclerotic effects achieved by administering said first and second pharmaceutical compositions separately and which second pharmaceutical composition comprises an amount of amlodipine or a pharmaceutically acceptable acid addition salt thereof and a pharmaceutically acceptable carrier or diluent, said first pharmaceutical composition comprising an amount of a statin or a pharmaceutically acceptable salt thereof and a pharmaceutically acceptable carrier or diluent; provided that said statin is not atorvastatin or a pharmaceutically acceptable salt thereof.

This invention is particularly directed to a composition, hereinafter termed “Composition KA”, of Composition K wherein said statin is simvastatin, pravastatin, rivastatin, mevastatin, fluindostatin, velostatin, fluvastatin, dalvastatin, dihydrocompactin, compactin or lovastatin; or a pharmaceutically acceptable salt of simvastatin, pravastatin, rivastatin, mevastatin, fluindostatin, velostatin, fluvastatin, dalvastatin, dihydrocompactin, compactin or lovastatin.

This invention is particularly directed to a composition, hereinafter termed “Composition KB”, of Composition KA wherein said statin is simvastatin, pravastatin, rivastatin, mevastatin, fluindostatin, velostatin, fluvastatin, dalvastatin, dihydrocompactin or compactin; or a pharmaceutically acceptable salt of simvastatin, pravastatin, rivastatin, mevastatin, fluindostatin, velostatin, fluvastatin, dalvastatin, dihydrocompactin or compactin.

This invention is more particularly directed to a composition, hereinafter termed “Composition KB”, of Composition KA wherein said second pharmaceutical composition comprises amlodipine besylate.

This invention is still more particularly directed to a composition, hereinafter termed “Composition KC” of Composition KB wherein said antiatherosclerotic effect is manifested by a slowing of the progression of atherosclerotic plaques.

This invention is still more particularly directed to a composition of Composition KC wherein said progression of atherosclerotic plaques is slowed in coronary arteries.

This invention is also particularly directed to a composition of Composition KC wherein said progression of atherosclerotic plaques is slowed in carotid arteries.

This invention is also particularly directed to a composition of Composition KC wherein said progression of atherosclerotic plaques is slowed in the peripheral arterial system.

This invention is also particularly directed to a composition, hereinafter termed “Composition KD”, of Composition KB wherein said antiatheroscierotic effect is manifested by a regression of atherosclerotic plaques.

This invention is more particularly directed to a composition of Composition KD wherein said regression of atherosclerotic plaques occurs in coronary arteries.

This invention is also more particularly directed to a composition of Composition KD wherein said regression of atherosclerotic plaques occurs in carotid arteries.

This invention is also more particularly directed to a composition of Composition KD wherein said regression of atherosclerotic plaques occurs in the peripheral arterial system.

This invention is also directed to a first pharmaceutical composition, hereinafter termed “Composition L”, for use with a second pharmaceutical composition for achieving an antiatherosclerotic effect in a mammal, which effect is greater than the sum of the antiatherosclerotic effects achieved by administering said first and second pharmaceutical compositions separately and which second pharmaceutical composition comprises an amount of a statin or a pharmaceutically acceptable salt thereof and a pharmaceutically acceptable carrier or diluent, said first pharmaceutical composition comprising an amount of amlodipine or a pharmaceutically acceptable acid addition salt thereof and a pharmaceutically acceptable carrier or diluent; provided that said statin is not atorvastatin or a pharmaceutically acceptable salt thereof.

This invention is particularly directed to a composition, hereinafter termed “Composition LA”, of Composition L wherein said statin is simvastatin, pravastatin, rivastatin, mevastatin, fluindostatin, velostatin, fluvastatin, dalvastatin, dihydrocompactin, compactin or lovastatin; or a pharmaceutically acceptable salt of simvastatin, pravastatin, rivastatin, mevastatin, fluindostatin, velostatin, fluvastatin, dalvastatin, dihydrocompactin, compactin or lovastatin.

This invention is particularly directed to a composition, hereinafter termed. “Composition LB”, of Composition LA wherein said statin is simvastatin, pravastatin, rivastatin, mevastatin, fluindostatin, velostatin, fluvastatin, dalvastatin, dihydrocompactin or compactin; or a pharmaceutically acceptable salt of simvastatin, pravastatin, rivastatin, mevastatin, fluindostatin, velostatin, fluvastatin, dalvastatin, dihydrocompactin or compactin.

This invention is more particularly directed to a composition, hereinafter termed “Composition LB”, of Composition LA comprising amlodipine besylate.

This invention is still more particularly directed to a composition, hereinafter termed “Composition LC”, of Composition LB wherein said antiatherosclerotic effect is manifested by a slowing of the progression of atherosclerotic plaques.

This invention is still more particularly directed to a composition of Composition LC wherein said progression of atherosclerotic plaques is slowed in coronary arteries.

This invention is still more particularly directed to a composition of Composition LC wherein said progression of atherosclerotic plaques is slowed in carotid arteries.

This invention is still more particularly directed to a composition of Composition LC wherein said progression of atherosclerotic plaques is slowed in the peripheral arterial system.

This invention is also particularly directed to a composition, hereinafter, termed “Composition LD”, of Composition LB wherein said antiatherosclerotic effect is manifested by a regression of atherosclerotic plaques.

This invention is still more particularly directed to a composition of Composition LD wherein said regression of atherosclerotic plaques occurs in coronary arteries.

This invention is still more particularly directed to a composition of Composition LD wherein said regression of atherosclerotic plaques occurs in carotid arteries.

This invention is still more particularly directed to a composition of Composition LD wherein said regression of atherosclerotic plaques occurs in the peripheral arterial system.

This invention is also directed to a first pharmaceutical composition, hereinafter termed “Composition M”, for use with a second pharmaceutical composition for achieving an antiatherosclerotic effect in a mammal, which effect is greater than the antiatherosclerotic effects achieved by administering said first or second pharmaceutical compositions separately and which second pharmaceutical composition comprises an amount of a statin or a pharmaceutically acceptable salt thereof and a pharmaceutically acceptable carrier or diluent, said first pharmaceutical composition comprising an amount of amlodipine or a pharmaceutically acceptable acid addition salt thereof and a pharmaceutically acceptable carrier or diluent; provided that said statin is not atorvastatin or a pharmaceutically acceptable salt thereof.

This invention is still more particularly directed to a composition of claim M comprising amlodipine besylate.

This invention is also directed to a first pharmaceutical composition, hereinafter termed “Composition N”, for use with a second pharmaceutical composition for achieving an antiatherosclerotic effect in a mammal, which effect is greater than the antiatherosclerotic effects achieved by administering said first or second pharmaceutical compositions separately and which second pharmaceutical composition comprises an amount of amlodipine or a pharmaceutically acceptable acid addition salt thereof and a pharmaceutically acceptable carrier or diluent, said first pharmaceutical composition comprising an amount of a statin or a pharmaceutically acceptable salt thereof and a pharmaceutically acceptable carrier or diluent; provided that said statin is not atorvastatin or a pharmaceutically acceptable salt thereof.

This invention is particularly directed to a composition, hereinafter termed “Composition NA”, of Composition N wherein said statin is simvastatin, pravastatin, rivastatin, mevastatin, fluindostatin, velostatin, fluvastatin, dalvastatin, dihydrocompactin, compactin or lovastatin; or a pharmaceutically acceptable salt of simvastatin, pravastatin, rivastatin, mevastatin, fluindostatin, velostatin, fluvastatin, dalvastatin, dihydrocompactin, compactin or lovastafin.

This invention is particularly directed to a composition of Composition NA wherein said statin is simvastatin, pravastatin, rivastatin, mevastatin, fluindostatin, velostatin, fluvastatin, dalvastatin, dihydrocompactin or compactin; or a pharmaceutically acceptable salt of simvastatin, pravastatin, rivastatin, mevastatin, fluindostatin, velostatin, fluvastatin, dalvastatin, dihydrocompactin or compactin.

This invention is also directed to a first pharmaceutical composition, hereinafter termed “Composition P”, for use with a second pharmaceutical composition for managing cardiac risk in a mammal at risk of suffering an adverse cardiac event, which effect is greater than the sum of the cardiac risk management effects achieved by administering said first and second pharmaceutical compositions separately and which second pharmaceutical composition comprises an amount of a statin or a pharmaceutically acceptable salt thereof and a pharmaceutically acceptable carrier or diluent, said first pharmaceutical composition comprising an amount of amlodipine or a pharmaceutically acceptable acid addition salt thereof and a pharmaceutically acceptable carrier or diluent, provided that said statin is not atorvastatin or a pharmaceutically acceptable salt thereof.

This invention is particularly directed to a composition, hereinafter termed “Composition PA” of Composition P wherein said statin is simvastatin, pravastatin, dalvastatin, mevastatin, fluindostatin, velostatin, fluvastatin, dalvastatin, dihydrocompactin, compactin or lovastatin; or a pharmaceutically acceptable salt of simvastatin, pravastatin, rivastatin, mevastatin, fluindostatin, velostatin, fluvastatin, dalvastatin, dihydrocompactin, compactin, lovastatin or pharmaceutically acceptable salts thereof.

This invention is particularly directed to a composition, hereinafter termed “Composition PB” of Composition PA wherein said statin is simvastatin, pravastatin, rivastatin, mevastatin, fluindostatin, velostatn, fluvastatin, dalvastatin, dihydrocompactin or compactin; or a pharmaceutically acceptable salt of simvastatin, pravastatin, rivastatin, mevastatin, fluindostatin, velostatin fluvastatin, dalvastatin, dihydrocompactin or compactin.

This invention is more particularly directed to a composition of Composition PA comprising amlodipine besylate.

This invention is also directed to a first pharmaceutical composition hereinafter termed “Composition Q” for use with a second pharmaceutical composition for managing cardiac risk in a mammal at risk of suffering an adverse cardiac event which effect is greater than the sum of the cardiac risk management effects achieved by administering said first and second pharmaceutical compositions separately and which second pharmaceutical composition comprises an amount of amlodipine or a pharmaceutically acceptable add addition salt thereof and a pharmaceutically acceptable carrier or diluent, said first pharmaceutical composition comprising an amount of a statin or a pharmaceutically acceptable salt thereof and a pharmaceutically acceptable carrier or diluent, provided that said statin is not atorvastatin or a pharmaceutically acceptable salt thereof.

This invention is particularly directed to a composition, hereinafter termed “Composition QA”, of Composition Q wherein said statin is simvastatin, pravastatin, rivastatin, mevastatin, fluindostatin, velostatin, fluvastatin, dalvastatin, dihydrocompactin, compactin or lovastatin; or a pharmaceutically acceptable salt of simvastatin, pravastatin, rivastatin, mevastatin, fluindostatin, velostatin, fluvastatin, dalvastatin, dihydrocompactin, compactin or lovastatin.

This invention is particularly directed to a composition of Composition QA wherein said statin is simvastatin, pravastatin, rivastatin, mevastatin, fluindostatin, velostatin, fluvastatin, dalvastatin, dihydrocompactin or compactin; or a pharmaceutically acceptable salt of simvastatin, pravastatin, rivastatin, mevastatin, fluindostatin, velostatin, fluvastatin, dalvastatin, dihydrocompactin or compactin.

This invention is more particularly directed to a composition of Composition QA wherein said second pharmaceutical composition comprises amlodipine besylate.

This invention is also directed to a first pharmaceutical composition, hereinafter termed “Composition R”, for use with a second pharmaceutical composition for managing cardiac risk in a mammal at risk of suffering an adverse cardiac event, which effect is greater than the cardiac risk management effects achieved by administering said first or second pharmaceutical compositions separately and which second pharmaceutical composition comprises an amount of a statin or a pharmaceutically acceptable salt thereof and a pharmaceutically acceptable carrier or diluent, said first pharmaceutical composition comprising an amount of amlodipine or a pharmaceutically acceptable acid addition salt thereof and a pharmaceutically acceptable carrier or diluent; provided that said statin is not atorvastatin or a pharmaceutically acceptable salt thereof.

This invention is still more particularly directed to a composition of Composition R comprising amlodipine besylate.

This invention is also directed to a first pharmaceutical composition, hereinafter termed “Composition S”, for use with a second pharmaceutical composition for managing cardiac risk in a mammal at risk of suffering an adverse cardiac event, which effect is greater than the cardiac risk management effects achieved by administering said first or second pharmaceutical compositions separately and which second pharmaceutical composition comprises an amount of amlodipine or a pharmaceutically acceptable acid addition salt thereof and a pharmaceutically acceptable carrier or diluent, said first pharmaceutical composition comprising an amount of a statin or a pharmaceutically acceptable salt thereof and a pharmaceutically acceptable carrier or diluent; provided that said statin is not atorvastatin or a pharmaceutically acceptable salt thereof.

This invention is particularly directed to a composition, hereinafter termed “Composition SA”, of Composition S wherein said statin is simvastatin, pravastatin rivastatin, mevastatin, fluindostatin, velostatin, fluvastatin, dalvastatin, dihydrocompactin, compactin or lovastatin; or a pharmaceutically acceptable salt of simvastatin, pravastatin, rivastatin, mevastatin, fluindostatin, velostatin, fluvastatin, dalvastatin, dihydrocompactin, compactin or lovastatin.

This invention is particularly directed to a composition of Composition SA wherein said statin is simvastatin, pravastatin, rivastatin, mevastatin, fluindostatin, velostatin, fluvastatin, dalvastatin, dihydrocompactin or compactin; or a pharmaceutically acceptable salt of simvastatin, pravastatin, rivastatin, mevastatin, fluindostatin, velostatin, fluvastatin, dalvastatin, dihydrocompactin or compactin.

This invention is also directed to a kit, hereinafter termed “Kit A”, for achieving a therapeutic effect in a mammal comprising:
a. an amount of amlodipine or a pharmaceutically acceptable acid addition salt thereof and a pharmaceutically acceptable carrier or diluent in a first unit dosage form;
b. an amount of a statin or a pharmaceutically acceptable salt thereof and a pharmaceutically acceptable carrier or diluent in a second unit dosage form; and
c. container means for containing said first and second dosage forms; provided that said statin is not atorvastatin or a pharmaceutically acceptable salt thereof.

This invention is particularly directed to a kit, hereinafter termed “Kit AA”, of Kit A wherein said statin is simvastatin, pravastatin, rivastatin, mevastatin, fluindostatin, velostatin, fluvastatin, dalvastatin, dihydrocompactin, compactin or lovastatin; or a pharmaceutically acceptable salt of simvastatin, pravastatin, rivastatin, mevastatin, fluindostatin velostatin, fluvastatin, dalvastatin, dihydrocompactin, compactin or lovastatin.

This invention is particularly directed to a kit, hereinafter termed “Kit AB”, of Kit AA wherein said statin is simvastatin, pravastatin, rivastatin, mevastatin, fluindostatin, velostatin, fluvastatin, dalvastatin, dihydrocompactin or compactin; or a pharmaceutically acceptable salt of simvastatin, pravastatin, rivastatin, mevastatin, fluindostatin, velostatin, fluvastatin, dalvastatin, dihydrocompactin or compactin.

This invention is more particularly directed to a kit, hereinafter “Kit AZ”, of Kit AA comprising amlodipine besylate.

This invention is also particularly directed to a kit of Kit A wherein said therapeutic effect is treatment of hypertension and hyperlipidemia.

This invention is also particularly directed to a kit of Kit A wherein said therapeutic effect is treatment of angina pectoris.

This invention is also particularly directed to a kit of Kit A wherein said therapeutic effect is management of cardiac risk.

This invention is also particularly directed to a kit, hereinafter termed “Kit AB” of Kit A wherein said therapeutic effect is treatment of atherosclerosis.

This invention is more particularly directed to a kit, hereinafter termed “Kit AC”, of Kit AB wherein said treatment of atherosclerosis slows the progression of atherosclerotic plaques.

This invention is still more particularly directed to a kit of Kit AC wherein said progression of atherosclerotic plaques is slowed in coronary arteries.

This invention is also more particularly directed to a kit of Kit AC wherein said progression of atherosclerotic plaques is slowed in carotid arteries.

This invention is also more particularly directed to a kit of Kit AC wherein said progression of atherosclerotic plaques is slowed, in the peripheral arterial system.

A kit, hereinafter termed “Kit AD” of Kit AB wherein said treatment of atherosclerosis causes the regression of atherosclerotic plaques.

This invention is still more particularly directed to a kit of Kit AD wherein said regression of atherosclerotic plaques occurs in coronary arteries.

This invention is also more particularly directed to a kit of Kit AD wherein said regression of atherosclerotic plaques occurs in carotid arteries.

This invention is also more particularly directed to a kit of Kit AD wherein said regression of atherosclerotic plaques occurs in the peripheral arterial system.

This invention is also directed to a kit, hereinafter termed “Kit AE”, of Kit AZ wherein said therapeutic effect is treatment of hypertension and hyperlipidemia.

This invention is also directed to a kit, hereinafter termed “Kit AF”, of Kit AZ wherein said therapeutic effect is treatment of angina pectoris.

This invention is also directed to a kit, hereinafter termed “Kit AG”, of Kit AZ wherein said therapeutic effect is treatment of cardiac risk.

This invention is also directed to a kit, hereinafter termed “Kit AH”, of Kit AZ wherein said therapeutic effect is treatment of atherosclerosis.

This invention is particularly directed to a kit, hereinafter termed “Kit AJ”, of Kit AH wherein said treatment of atherosclerosis slows the progression of atherosclerotic plaques.

This invention is also more particularly directed to a kit of Kit AJ wherein said progression of atherosclerotic plaques is slowed in coronary arteries.

This invention is also more particularly directed to a kit of Kit AJ wherein said progression of atherosclerotic plaques is slowed in carotid arteries.

This invention is also more particularly directed to a kit of Kit AJ wherein said progression of atherosclerotic plaques is slowed in the peripheral arterial system.

This invention is more particularly directed to a kit, hereinafter termed “Kit AK”, of Kit AH wherein said treatment of atherosclerosis causes the regression of atherosclerotic plaques.

This invention is still more particularly directed to a kit of Kit AK wherein said regression of atherosclerotic plaques occurs in coronary arteries.

This invention is also more particularly directed to a kit of Kit AK wherein said regression of atherosclerotic plaques occurs in carotid arteries.

This invention is also more particularly directed to a kit of Kit AK wherein said regression of atherosclerotic plaques occurs in the peripheral arterial system.

This invention is also directed to a method, hereinafter termed “Method A”, for treating a mammal in need of therapeutic treatment comprising administering to said mammal
(a) an amount of a first compound, said first compound being amlodipine or a pharmaceutically acceptable acid addition salt thereof; and
(b) an amount of a second compound, said second compound being statin or a pharmaceutically acceptable salt thereof;
   wherein said first compound and said second compound are each optionally and independently administered together with a pharmaceutically acceptable carrier or diluent;
   provided that said statin is not atorvastatin or a pharmaceutically acceptable salt thereof.

This invention is particularly directed to a method, hereinafter termed “Method AK”, of Method A wherein said statin is simvastatin, pravastatin, rivastatin, mevastatin, fluindostatin, velostatin, fluvastatin, dalvastatin, dihydrocompactin, compactin or lovastatin; or a pharmaceutically acceptable salt of simvastatin, pravastatin, rivastatin, mevastatin, fluindostatin, velostatin, fluvastatin, dalvastatin, dihydrocompactin, compactin, lovastatin or pharmaceutically acceptable salts thereof.

This invention is particularly directed to a method, hereinafter termed “Method AB”, of Method AA wherein said statin is simvastatin, pravastatin, rivastatin, mevastatin, fluindostatin, velostatin, fluvastatin, dalvastatin, dihydrocompactin or compactin; or a pharmaceutically acceptable salt of simvastatin, pravastatin, rivastatin, mevastatin, fluindostatin, velostatin, fluvastatin, dalvastatin, dihydrocompactin or compactin.

This invention is more particularly directed to a method, hereinafter termed “Method AB”, of Method AA comprising amlodipine besylate.

This invention is also particularly directed to a method, hereinafter termed “Method AC”, of Method A wherein said first compound and said second compound are administered simultaneously.

This invention is also particularly directed to a method hereinafter termed “Method AD”, of Method A wherein said first compound and said second compound are administered sequentially in either order.

This invention is more particularly directed to a method, hereinafter termed “Method AE”, of Method AB wherein said first compound and said second compound are administered simultaneously.

This invention is also more particularly directed to a method, hereinafter termed “Method AF”, of Method AB wherein said first compound and said second compound are administered sequentially in either order.

This invention is also particularly directed to a method, hereinafter termed “Method AG”, of Method A wherein said therapeutic treatment comprises antihypertensive treatment and antihyperlipidemic treatment.

This invention is also particularly directed to a method of Method AE wherein said therapeutic treatment comprises antihypertensive treatment and antihyperlipidemic treatment.

This invention is also particularly directed to a method of Method AF wherein said therapeutic treatment comprises antihypertensive treatment and antihypedipidemic treatment.

This invention is also particularly directed to a method of Method A wherein said therapeutic treatment comprises antianginal treatment.

This invention is also particularly directed to a method of Method AE wherein said therapeutic treatment comprises antianginal treatment.

This invention is also particularly directed to a method of Method AF wherein said therapeutic treatment comprises anbanginal treatment.

This invention is also particularly directed to a method of Method A wherein said therapeutic treatment comprises cardiac risk management.

This invention is also particularly directed to a method of Method AE wherein said therapeutic treatment comprises cardiac risk management.

This invention is also particularly directed to a method of Method AF wherein said therapeutic treatment comprises cardiac risk management.

This invention is also particularly directed to a method of Method A wherein said therapeutic treatment comprises antiatherosclerotic treatment.

This invention is also particularly directed to a method of Method AE wherein said therapeutic treatment comprises antiatherosclerotic treatment.

This invention is also particularly directed to a method of Method AF wherein said therapeutic treatment comprises antiatherosclerotic treatment.

Amlodipine is a racemic compound due to the symmetry at position 4 of the dihydropyridine ring. The R and S enantiomers may be prepared as described by Arrowsmith et al., J. Med. Chem., 1986, 29, 1696. The calcium channel blocking activity of amlodipine is substantially confined to the S(−) isomer and to the racemic mixture containing the R(+) and S(−) forms. (see International Patent Application Number PCT/EP94/02697). The R(+) isomer has little or no calcium channel blocking activity. However, the R(+) isomer is a potent inhibitor of smooth muscle cell migration. Thus, the R(+) isomer is useful in the treatment or prevention of atherosclerosis. (see International Patent Application Number PCT/EP95/00847). Based on the above, a skilled person could choose the R(+) isomer, the S(−) isomer or the racemic mixture of the R(+) isomer and the S(−) isomer for use in the combination of this invention.

Where used herein, the term “cardiac risk” means the likelihood that a subject will suffer a future adverse cardiac event such as, e.g., myocardial infarction, cardiac arrest, cardiac failure, cardiac ischaemia. Cardiac risk is calculated using the Framingham Risk Equation as set forth above. The term “cardiac risk management” means that the risk of future adverse cardiac events is substantially reduced.

### DETAILED DESCRIPTION OF THE INVENTION

The combinations of this invention comprise two active components: amlodipine or a pharmaceutically acceptable acid addition salt thereof and a statin or a pharmaceutically acceptable salt thereof. The combination of this invention may also include a pharmaceutically acceptable carrier or diluent.

Amlodipine is a potent calcium channel blocker and as such has utility in the treatment of hypertension. Amlodipine is prepared as described in U.S. Pat. No. 4,572,909, which is incorporated herein by reference. Amlodipine besylate, which is currently sold as Norvasc®, may be prepared as described in U.S. Pat. No. 4,879,303, which is incorporated herein by reference. Amlodipine, amlodipine besylate and other pharmaceutically acceptable acid addition salts of amlodipine are potent and long lasting calcium channel blockers. Other acid addition salts of amlodipine may be prepared by reacting the free base form of amlodipine with the appropriate add. When the salt is of a monobasic acid (e.g., the hydrochloride, the hydrobromide, the, p-toluenesulfonate, the acetate), the hydrogen form of a dibasic acid (e.g., the hydrogen sulfate, the succinate) or the dihydrogen form of a tribasic acid (e.g., the dihydrogen phosphate, the citrate), at least one molar equivalent and usually a molar excess of the add is employed. However, when such salts as the sulfate, the hemisuccinate, the hydrogen phosphate or the phosphate are desired, the appropriate and exact chemical equivalents of add will generally be used. The free base of amlodipine and the acid are usually combined in a co-solvent from which the desired salt precipitates, or can be otherwise isolated by concentration and/or addition of a non-solvent.

The other active component of the combinations of this invention is a statin. The term “statin” where used in the specification and the appendant claims, is synonymous with the terms “3-hydroxy-3-methylglutaryl-Coenzyme A reductase inhibitor” and “HMG-CoA reductase inhibitor.” These three terms are used interchangeably throughout the specification and appendant claims. As the synonyms suggest, statins are inhibitors of 3hydroxy-3-methylglutaryl-Coenzyme A reductase and as such are effective in lowering the level of blood plasma cholesterol. Statins and pharmaceutically acceptable salts thereof are particularly useful in lowering low density lipoprotein cholesterol (LDL-C) levels in mammals and particularly in humans.

The HMG-CoA reductase inhibitors suitable for use herein include, but are not limited to, simvastatin, pravastatin, rivastatin, mevastatin, fluindostatin, velostatin, fluvastatin, dalvastatin, dihydrocompactin, compactin or lovastatin; or a pharmaceutically acceptable salt of simvastatin, pravastatin, rivastatin, mevastatin, fluindostatin, velostatin, fluvastatin, dalvastatin, dihydrocompactin, compactin, lovastatin or pharmaceutically acceptable salts thereof. However, it is to be noted that atorvastatin or a pharmaceutically acceptable salt thereof is not within the scope of this disclosure.

The statins disclosed herein are prepared by methods well known to those skilled in the art. Specifically, simvastatin may be prepared according to the method disclosed in U.S. Pat. No. 4,444,784, which is incorporated herein by reference. Pravastatin may be prepared according to the method disclosed in U.S. Pat. No. 4,346,227, which is incorporated herein by reference. Cerivastatin may be prepared according to the method disclosed in U.S. Pat. No. 5,502,199, which is incorporated herein by reference. Cerivastatin may alteratively be prepared according to the method disclosed in European Patent Application Publication No. EP617019. Mevastatin may be prepared according to the method disclosed in U.S. Pat. No. 3,983,140, which is incorporated herein by reference. Velostatin may be prepared according to the methods disclosed in U.S. Pat. No. 4,448,784 and U.S. Pat. No. 4,450,171, both of which are incorporated herein by reference. Fluvastatin may be prepared according to the method disclosed in U.S. Pat. No. 4,739,073, which is incorporated herein by reference. Compactin may be prepared according to the method disclosed in U.S. Pat. No. 4,804,770, which is incorporated herein by reference. Lovastatin may be prepared according to the method disclosed in U.S. Pat. No. 4,231,938, which is incorporated herein by reference. Dalvastatin may be prepared according to the method disclosed in European Patent Application Publication No. 738510 A2. Fluindostatin may be prepared according to the method disclosed in European Patent Application Publication No. 363934 A1. Dihydrocompactin may be prepared according to the method disclosed in U.S. Pat. No. 4,450,171, which is incorporated herein by reference.

It will be recognized that certain of the above statins contain either a free carboxylic acid or a free amine group as part of the chemical structure. Further, certain statins within the scope of this invention contain lactone moieties which exist in equilibrium with the free carboxylic acid form. These factories can be maintained as carboxylates by preparing pharmaceutically acceptable salts of the lactone. Thus, this invention includes pharmaceutically acceptable salts of those carboxylic acids or amine groups. The expression “pharmaceutically acceptable salts” includes both pharmaceutically acceptable acid addition salts and pharmaceutically acceptable cationic salts. The expression “pharmaceutically-acceptable cabonic salts” is intended to define but is not limited to such salts as the alkali metal salts, (e.g. sodium and potassium), alkaline earth metal salts (e.g. calcium and magnesium), aluminum salts, ammonium salts, and salts with organic amines such as bermathine (N,N′-dibenzylethylenediamine), choline, dimethylamine, ethylenediamine, meglumine (N-methylglucamine), beneftamine (N-benzylphenethylamine), diethylamine, piperazine, tromethamine (2-amino-2-hydroxymethyl-1,3propanediol) and procaine. The expression “pharmaceutically-acceptable acid addition salts” is intended to define but is not limited to such salts as the hydrochloride, hydrobromide, sulfate, hydrogen sulfate, phosphate, hydrogen phosphate, dihydrogenphosphate, acetate, succinate, citrate, methanesulfonate (mesylate) and p-toluenesulfonate (tosylate) salts.

The pharmaceutically-acceptable cationic salts of statins containing free carboxylic acids may be readily prepared by reacting the free acid form of the statin with an appropriate base, usually one equivalent, in a co-solvent. Typical bases are sodium hydroxide, sodium methoxide, sodium ethoxide, sodium hydride, potassium methoxide, magnesium hydroxide, calcium hydroxide, benzathine, choline, diethanolaminei piperazine and tromethamine. The salt is isolated by concentration to dryness or by addition of a non-solvent. In many cases, salts are preferably prepared by mixing a solution of the acid with a solution of a different salt of the cation (sodium or potassium ethylhexanoate, magnesium oleate), employing a solvent (e.g., ethyl acetate) from which the desired catonic salt precipitates, or can be otherwise isolated by concentration and/or addition of a non-solvent.

The pharmaceutically acceptable acid addition salts of statins containing free amine groups may be readily prepared by reacting the free base form of the statin with the appropriate acid. When the salt is of a monobasic acid (e.g., the hydrochloride, the hydrobromide, the p-toluenesulfonate, the acetate), the hydrogen form of a dibasic acid (e.g., the hydrogen sulfate, the succinate) or the dihydrogen form of a tribasic acid (e.g., the dihydrogen phosphate, the citrate), at least one molar equivalent and usually a molar excess of the acid is employed. However when such salts as the sulfate, the hemisuccinate, the hydrogen phosphate or the phosphate are desired, the appropriate and exact chemical equivalents of acid will generally be used. The free base and the acid are usually combined in a co-solvent from which the desired salt precipitates, or can be otherwise isolated by concentration and/or addition of a non-solvent.

In addition, amlodipine and pharmaceutically acceptable acid addition salts thereof may occur as hydrates or solvates. Further, the statins of the instant invention and the pharmaceutically acceptable salts of the statins of the instant invention may also occur as hydrates or solvates. Said hydrates and solvates are also within the scope of the invention.

The pharmaceutical combinations and methods of this invention are all adapted to therapeutic use as agents in the treatment of atherosclerosis, angina pectoris, and a condition characterized by the presence of both hypertension and hyperlipidemia in mammals, particularly humans. Further, since these diseases and conditions are closely related to the development of cardiac disease and adverse cardiac conditions, these combinations and methods, by virtue of their action as antiatherosclerotics, antianginals, antihypertensives and antihyperlipidemics, are useful in the management of cardiac risk in subjects at risk of developing adverse cardiac conditions and in subjects at risk of suffering adverse cardiac events.

The utility of the compounds of the present invention as medical agents in the treatment of atherosclerosis in mammals (e.g. humans) is demonstrated by the activity of the compounds of this invention in conventional assays and the clinical protocol described below:

### Effect of Amlodipine and a Statin, Alone and in Combination, on the Treatment of Atherosclerosis

This study is a prospective randomized evaluation of the effect of a combination of amlodipine or a pharmaceutically acceptable salt thereof and a statin on the progression/regression of coronary and carotid artery disease. The study is used to show that a combination of amlodipine or a pharmaceutically acceptable acid addition salt and a statin is effective in slowing or arresting the progression or causing regression of existing coronary artery disease (CAD) as evidenced by changes in coronary angiography or carotid ultrasound, in subjects with established disease.

This study is an angiographic documentation of coronary artery disease carried out as a double-blind, placebo-controlled trial of a minimum of about 500 subjects and preferably of about 780 to about 1200 subjects. It is especially preferred to study about 1200 subjects in this study. Subjects are admitted into the study after satisfying certain entry criteria set forth below.

Entry criteria: Subjects accepted for entry into this trial must satisfy certain criteria. Thus the subject must be an adult, either male or female, aged 18-80 years of age in whom coronary angiography is clinically indicated. Subjects mill have angiographic presence of a significant focal lesion such as 30% to 50% on subsequent evaluation by quantitative coronary angiography (QCA) in a minimum of one segment (non-PTCA, non-bypassed or non-MI vessel) that is judged not likely to require intervention over the next 3 years. It is required that the segments undergoing analysis have not been interfered with. Since percutaneous transluminal cardiac angioplasty (PTCA) interferes with segnerdts by the insertion of a balloon catheter, non-PTCA segments are required for analysis. It is also required that the segments to be analyzed have not suffered a thrombotic event, such as a myocardial infarct (MI). Thus the requirement for non-MI vessels. Segments that will be analyzed include: left main, proximal, mid and distal left anterior descending, first and second diagonal branch, proximal and distal left circumflex, first or largest space obtuse marginal, proximal, mid and distal right coronary artery. Subjects will have an ejection fraction of greater than 30% determined by catheterization or radionudide ventriculography or ECHO cardiogram at the time of the qualifying angiogram or within the previous three months of the acceptance of the qualifying angiogram provided no intervening event such as a thrombotic event or procedure such as PTCA has occurred.

Generally, due to the number of patents and the physical limitations of any one facility, the study is carried out at multiple sites. At entry into the study, subjects undergo quantitative coronary angiography as well as B-mode carotid artery ultrasonography and assessment of carotid arterial compliance at designated testing centers. This establishes baselines for each subject. Once admitted into the test subjects are randomized to recene amlodipine besylate (10 mgs) and placebo or a statin (dose is dependent upon the particular statin used, however generally 80 mgs will be used at first) and placebo or amlodipine besylate (10 mgs) and a statin (80 mgs). It will be recognized by a skilled person that the free base form or other salt forms of amlodipine besylate or the free base form or other salt forms of the statin may be used in this invention. Calculation of the dosage amount for these other forms of the statin and amlodipine besylate is easily accomplished by performing a simple ratio relative to the molecular weights of the species involved. The amount of amlodipine may be varied as required. Generally, a subject will star out taking 10 mg and the amount will be titrated down to as litte as 5 mg as determined by the clinical physician. The amount of the statin will similarly be titrated down from 80 mg if it is determined by the physician to be in the best interests of the subject. The subjects are monitored for a one to three year period, generally three years being preferred. B-mode carotid ultrasound assessment of carotid artery atherosclerosis and compliance are performed at regular intervals throughout the study.

Generally, six month intervals are suitable. Typically this assessment is performed using B-mode ultrasound equipment. However, a person skilled in the art may use other methods of performing this assessment. Coronary angiography is performed at the conclusion of the one to three year treatment period. The baseline and post-treatment angiograms and the intervening carotid artery B-mode ultrasonograms are evaluated for new lesions or progression of existing atherosclerotic lesions. Artertal compliance measurements are assessed for changes from baseline and over the 6-month evaluation periods.

The primary objective of this study is to show that the combination of amlodipine or a pharmaceutically acceptable acid addition salt and a statin reduces the progression of atherosclerotic lesions as measured by quantitative coronary angiography (QCA) in subjects with clinical coronary artery disease. QCA measures the opening in the lumen of the arteries measured.

The primary endpoint of the study is the change in the average mean segment diameter of the coronary artery tree. Thus, the diameter of an arterial segment is measured at various portions along the length of that segment. The average diameter of that segment is then determined. After the average segment diameter of many segments has been determined, the average of all segment averages is determined to arrive at the average mean segment diameter. The mean segment diameter of subjects taking a statin and amlodipine or a pharmaceutically acceptable acid addition salt will decline more slowly, will be halted completely, or there will be an increase in the mean segment diameter. These results represent slowed progression of atherosclerosis, halted progression of atherosclerosis and regression of atherosclerosis, respectively.

The secondary objective of this study is that the combination of amiodipine or a pharmaceutically acceptable add addition salt and a statin reduces the rate of progression of atherosclerosis in the carotid arteries as measured by the slope of the maximum intimal-medial thickness measurements averaged over 12 separate wan segments (Mean Max) as a function of time, more than does amlodipine or a pharmaceutically acceptable acid addition salt or a stain alone. The intinal-medial thickness of subjects taking a statin and amlodipine or a pharmaceutically acceptable salt thereof will increase more slowly, will cease to increase or will decrease. These results represent slowed progression of atherosclerosis, halted progression of atherosclerosis and regression of atherosclerosis, respectively. Further, these results may be used to facilitate dosage determinations.

The utility of the compounds of the present invention as medical agents in the treatment of angina pectoris in mammals (e.g., humans) is demonstrated by the activity of the compounds of this invention in conventional assays and the clinical protocol described below:

### Effect of Amlodipine and a Statin, Alone and in Combination, on the Treatment of Angina

This study is a double blind, parallel arm, randomized study to show the effectiveness of amlodipine or a pharmaceutically acceptable acid addition salt thereof and a statin given in combination in the treatment of symptomatic angina.

Entry criteria: Subjects are males or females between 18 and 80 years of age with a history of typical chest pain associated with one of the following objective evidences of cardiac ischemia: (1) stress test segment elevation of about one millimeter or more from the ECG; (2) positive treadmill stress test; (3) new wall motion abnormality on ultrasound; or (4) coronary angiogram with a significant qualifying stenosis. Generally a stenosis of about 30-50% is considered to be significant.

Each subject is evaluated for about ten to thirty-two weeks. At least ten weeks are generally required to complete the study. Sufficient subjects are used in this screen to ensure that about 200 to 800 subjects and preferably about 400 subject are evaluated to complete the study. Subjects are screened for compliance with the entry criteria, set forth below, during a four week run in phase. After the screening criteria are met, subjects are washed out from their current anti-anginal medication and stabilized on a long acting nitrate such as nitroglycerine, isosorbide-5-mononitrate or isosorbide dinitrate. The term “washed out”, when used in connection with this screen, means the withdrawal of current anti-anginal medication so that substantially all of said medication is eliminated from the body of the subject. A period of eight weeks is preferably allowed for both the wash out period and for the establishment of the subject on stable doses of said nitrate. Subjects having one or two attacks of angina per week while on stable doses of long acting nitrate are generally permitted to skip the wash out phase. After subjects are stabilized on nitrates, the subjects enter the randomization phase provided the subjects continue to have either one or two angina attacks per week. In the randomization phase, the subjects are randomly placed into one of the four arms of the study set forth below. After completing the wash out phase, subjects in compliance with the entry criteria undergo twenty four hour ambulatory electrocardigram (ECG) such as Holter monitoring, exercise stress testing such as a treadmill and evaluation of myocardial perfusion using PET (photon emission tomography) scanning to establish a baseline for each subject. When conducting a stress test, the speed of the treadmill and the gradient of the treadmill can be controlled by a technician. The speed of the treadmill and the angle of the gradient are generally increased during the test. The time intervals between each speed and gradient increase is generally determined using a modified Bruce Protocol.

After the baseline investigations have been completed, subjects are initiated on one of the following four arms of the study: (1) placebo; (2) a statin (about 2.5 mg to about 160 mg); (3) amlodipine besylate (about 2.5 mg to about 20 mg); or (4) a combination of the above doses of amlodipine besylate and a statin together. The subjects are then monitored for two to twenty four weeks. It will be recognized by a skilled person that the free base form or other salt forms of amlodipine besylate or the free base form or other salt forms of the statin may be used in this invention. Calculation of the dosage amount for these other forms of the statin and amlodipine besylate is easily accomplished by performing a simple ratio relative to the molecular weights of the species involved.

After the monitoring period has ended, subjects will undergo the following investigations: (1) twenty four hour ambulatory ECG, such as Holter monitoring; (2) exercise stress testing (e.g. treadmill using said modified Bruce Protocol); and (3) evaluation of myocardial perfusion using PET scanning. Patients keep a diary of painful ischemic events and nitroglycerine consumption. It is generally desirable to have an accurate record of the number of anginal attacks suffered by the patient during the duration of the test. Since a patient generally takes nitroglycerin to ease the pain of an anginal attack, the number of times that the patient administers nitroglycerine provides a reasonably accurate record of the number of anginal attacks.

To demonstrate the effectiveness and dosage of the drug combination of this invention, the person conducting the test will evaluate the subject using the tests described. Successful treatment will yield fewer instances of ischemic events as detected by ECG, will allow the subject to exercise longer or at a higher intensity level on the treadmill, or to exercise without pain on the treadmill, or will yield better perfusion or fewer perfusion defects on photoemission tomography (PET).

The utility of the compounds of the present invention as medical agents in the treatment of hypertension and hyperlipidemia in mammals (e.g., humans) suffering from a combination of hypertension and hyperlipidemia is demonstrated by the activity of the compounds of this invention in conventional assays and the clinical protocol described below.

### Effect of Amlodipine and a Statin, Alone and in Combination, on the Treatment of Subjects Having Both Hypertension and Hyperlipidemia

This study is a double blind, parallel arm, randomized study to show the effectiveness of amlodipine or a pharmaceutically acceptable acid addition salt thereof and a statin given in combination in controlling both hypertension and hyperlipidemia in subjects who have mild, moderate, or severe hypertension and hyperlipidemia.

Each subject is evaluated for 10 to 20 weeks and preferably for 14 weeks. Sufficient subjects are used in this screen to ensure that about 400 to 800 subjects are evaluated to complete the study.

Entry criteria: Subjects are male or female adults between 18and 80 years of age having both hyperlipidemia and hypertension. The presence of hyperlipidemia is evidenced by evaluation of the low density lipoprotein (LDL) level of the subject relative to certain positive risk factors. If the subject has no coronary heart disease (CHD) and has less than two positive risk factors, then the subject is considered to have hyperlipidemia which requires drug therapy if the LDL of the subject is greater than or equal to 190. If the subject has no CHD and has two or more positive risk factors, then the subject is considered to have hyperlipidemia which requires drug therapy if the LDL of the subject is greater than or equal to 160. If the subject has CHD, then the subject is considered to have hyperlipidemia if the LDL of the subject is greater than or equal to 130.

Positive risk factors include (1) male over 45, (2) female over 55 wherein said female is not undergoing hormone replacement therapy (HRT), (3) family history of premature cardiovascular disease, (4) the subject is a current smoker, (5) the subject has diabetes, (6) an HDL of less than 45, and (7) the subject has hypertension. An HDL of greater than 60 is considered a negative risk factor and will offset one of the above mentioned positive risk factors.

The presence of hypertension is evidenced by a sitting diastolic blood pressure (BP) of greater than 90 or sitting systolic BP of greater than 140. All blood pressures are generally determined as the average of three measurements taken five minutes apart.

Subjects are screened for compliance with the entry criteria set forth above. After all screening criteria are met, subjects are washed out from their current antihypertensive and lipid lowering medication and are placed on the NCEP ATP II Step 1 diet. The NCEP ATP II (adult treatment panel, 2nd revision) Step 1 diet sets forth the amount of saturated and unsaturated fat which can be consumed as a proportion of the total caloric intake. The term “washed out” where used in connection with this screen, means the withdrawal of current antihypertensive and lipid lowering medication so that substantially all of said medication is eliminated from the body of the subject. Newly diagnosed subjects generally remain untreated until the test begins. These subjects are also placed on the NCEP Step 1 diet. After the four week wash out and diet stabilization period, subjects undergo the following baseline investigations: (1) blood pressure and (2) fasting lipid screen. The fasting lipid screen determines baseline lipid levels in the fasting state of a subject. Generally, the subject abstains from food for twelve hours, at which time lipid levels are measured.

After the baseline investigations are performed subjects are started on one of the following: (1) a fixed dose of amlodipine besylate, generally about 2.5 to 10 mg; (2) a fixed dose of a statin, generally about 2.5 mg to about 160 mg; or (3) a combination of the above doses of amlodipine besylate and a statin together. It will be recognized by a skilled person that the free base form or other salt forms of amlodipine besylate or the free base form or other salt forms of the statin may be used in this invention. Calculation of the dosage amount for these other forms of the statin and amlodipine besylate is easily accomplished by performing a simple ratio relative to the molecular weights of the species involved. Subjects remain on these doses for a minimum of six weeks, and generally for no more than eight weeks. The subjects return to the testing center at the conclusion of the six to eight weeks so that the baseline evaluations can be repeated. The blood pressure of the subject at the conclusion of the study is compared with the blood pressure of the subject upon entry. The lipid screen measures the total cholesterol, LDL-cholesterol, HDL-cholesterol, triglycerides, apoB, VLDL (very low density lipoprotein) and other components of the lipid profile of the subject. Improvements in the values obtained after treatment relative to pretreatment values indicate the utility of the drug combination.

The utility of the compounds of the present invention as medical agents in the management of cardiac risk in mammals (e.g., humans) at risk for an adverse cardiac event is demonstrated by the activity of the compounds of this invention in conventional assays and the clinical protocol described below:

### Effects of Amlodipine and a Statin, Alone and in Combination, on Subjects at Risk of Future Cardiovascular Events

This study is a double blind, parallel arm, randomized study to demonstrate the effectiveness of amlodipine or a pharmaceutically acceptable acid addition salt and a statin given in combination in reducing the overall calculated risk of future events in subjects who are at risk for having future cardiovascular events. This risk is calculated by using the Framingham Risk Equation. A subject is considered to be at risk of having a future cardiovascular event if that subject is more than one standard deviation above the mean as calculated by the Framingham Risk Equation. The study is used to evaluate the efficacy of a fixed combination of amlodipine or a pharmaceutically acceptable acid addition salt and a statin in controlling cardiovascular risk by controlling both hypertension and hyperlipidemia in patients who have both mild to moderate hypertension and hyperlipidemia.

Each subject is evaluated for 10 to 20 weeks and preferably for 14 weeks. Sufficient subjects are recruited to ensure that about 400 to 800 subjects are evaluated to complete the study.

Entry criteria: Subjects included in the study are male or female adult subjects between 18 and 80 years of age with a baseline five year risk which risk is above the median for said subjects age and sex, as defined by the Framingham Heart Study, which is an ongoing prospective study of adult men and women showing that certain risk factors can be used to predict the development of coronary heart disease. The age, sex, systolic and diastolic blood pressure, smoking habit, presence or absence of carbohydrate intolerance, presence or absence of left ventricular hypertrophy, serum cholesterol and high density lipoprotein (HDL) of more than one standard deviation above the norm for the Framingham Population are all evaluated in determining whether a patient is at risk for adverse cardiac event. The values for the risk factors are inserted into the Framingham Risk equation and calculated to determine whether a subject is at risk for a future cardiovascular event.

Subjects are screened for compliance with the entry criteria set forth above. After all screening criteria are met, patients are washed out from their current antihypertensive and lipid lowering medication and any other medication which will impact the results of the screen. The patients are then placed on the NCEP ATP II Step 1 diet, as described above. Newly diagnosed subjects generally remain untreated until the test begins. These subjects are also placed on the NCEP ATP II Step 1 diet. After the four week wash out and diet stabilization period, subjects undergo the following baseline investigations: (1) blood pressure; (2) fasting; (3) lipid screen; (4) glucose tolerance test; (5) ECG; and (6) cardiac ultrasound. These tests are carried out using standard procedures well known to persons skilled in the art. The ECG and the cardiac ultrasound are generally used to measure the presence or absence of left ventricular hypertrophy.

After the baseline investigations are performed patients will be started on one of the following: (1) a fixed dose of amlodipine besylate(about 2.5 to 10 mg); (2) a fixed dose of a statin (about 2.5 mg to about 160 mg); or (3) the combination of the above doses of amlodipine besylate and a statin. Patients are kept on these doses and are asked to return in six to eight weeks so that the baseline evaluations can be repeated. At this time the new values are entered into the Framingham Risk equation to determine whether the subject has a lower, greater or no change in the risk of future cardiovascular event.

The above assays demonstrating the effectiveness of amodipine or pharmaceutically acceptable acid addition salts thereof and atorvastatin or pharmaceutically acceptable salts thereof in the treatment of angina pectoris, atherosclerosis, hypertension and hyperlipidemia together, and the management of cardiac risk, also provide a means whereby the activities of the compounds of this invention can be compared between themselves and with the activities of other known compounds. The results of these comparisons are useful for determining dosage levels in mammals, including humans, for the treatment of such diseases.

The following dosage amounts and other dosage amounts set forth elsewhere in this specification and in the appendant claims are for an average human subject having a weight of about 65 kg to about 70 g. The skilled practitioner will readily be able to determine the dosage amount required for a subject whose weight falls outside the 65 kg to 70 kg range, based upon the medical history of the subject and the presence of diseases, e.g., diabetes, in the subject. All doses set forth herein, and in the appendant claims, are daily doses.

In general, in accordance with this invention, amlodipine is generally administered in a dosage of about 2.5 mg to about 20 mg. Preferably, amlodipine is administered in a dosage of about 5 mg to about 10 mg. It will be recognized by a skilled person that the free base form or other salt forms of amlodipine besylate may be used in this invention. Calculation of the dosage amount for these other forms of or the free base form or other salt forms of amlodipine besylate is easily accomplished by performing a simple ratio relative to the molecular weights of the species involved.

In general, in accordance with this invention, the above statins are administered in the following dosage amounts:

Simvastatin, generally about 2.5 mg to about 160 mg and preferably about 10 mg to about 40 mg;

pravastatin, generally about 2.5 mg to about 160 mg and preferably about 10 mg to about 40 mg;

cerivastatin, generally about 25 μg to about 5 mg and preferably about 1 mg to about 3.2 mg;

fluvastatin, generally about 2.5 mg to about 160 mg and preferably about 20 mg to about 80 mg; and

lovastafin, generally about 2.5 mg to about 160 mg and preferably about 10 mg to about 80 mg.

It will be recognized by a skilled person that the free base form or other salt forms of the above statins may be used in this invention. Calculation of the dosage amount for these other forms of or the free base form or other salt forms said statins is easily accomplished by performing a simple ratio relative to the molecular weights of the species involved.

The compounds of the present invention are generally administered in the form of a pharmaceutical composition comprising at least one of the compounds of this invention together with a pharmaceutically acceptable carrier or diluent. Thus, the compounds of this inventions can be administered either individually or together in any conventional oral, parenteral or transdermal dosage form.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixers are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

The combinations of this invention may also be administered in a controlled release formulation such as a slow release or a fast release formulation. Such controlled release formulations of the combination of this invention may be prepared using methods well known to those skilled in the art. The method of administration will be determined by the attendant physician or other person skilled in the art after an evaluation of the subject's condition and requirements. The generally preferred formulation of amlodipine is Norvasc®.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

Methods of preparing various pharmaceutical compositions with a certain amount of active ingredient are known, or will be apparent in light of this disclosure, to those skilled in this art. For examples, see Remington's Pharmaceutical Sciences, Mack Publishing Company, Easter, Pa., 15th Edition (1975).

Pharmaceutical compositions according to the invention may contain 0.1%-95% of the compound(s) of this invention, preferably 1%-70%. In any event, the composition or formulation to be administered will contain a quantity of a compound(s) according to the invention in an amount effective to treat the condition or disease of the subject being treated.

Since the present invention relates to the treatment of diseases and conditions with a combination of active ingredients which may be administered separately, the invention also relates to combining separate pharmaceutical compositions in kit form. The kit includes two separate pharmaceutical compositions: amlodipine or a pharmaceutically acceptable acid addition salt thereof and a statin or a pharmaceutically acceptable salt thereof. The kit includes container means for containing the separate compositions such as a divided bottle or a divided foil packet, however, the separate compositions may also be contained within a single, undivided container. Typically the kit includes directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

It should be understood that the invention is not limited to the particular embodiments described herein, but that various changes and modifications may be made without departing from the spirit and scope of this novel concept as defined by the following claims.

1. A pharmaceutical composition comprising:
a. an amount of amlodipine or a pharmaceutically acceptable acid addition salt thereof;
b. an amount of a statin or a pharmaceutically acceptable salt thereof; and
c. a pharmaceutically acceptable carrier or diluent;
provided that said statin is not atorvastatin or a pharmaceutically acceptable salt thereof.

2. A pharmaceutical composition of claim 1 wherein said statin is simvastatin, pravastatin, rivastatin, mevastatin, fluindostatin, velostatin, fluvastatin, dalvastatin, dihydrocompactin, compactin or lovastatin; or a pharmaceutically acceptable salt of simvastatin, pravastatin, rivastatin, mevastatin, fluindostatin, velostatin, fluvastatin, dalvastatin, dihydrocompactin, compactin or lovastatin.

3. A pharmaceutical composition of claim 2 wherein said statin is simvastatin, pravastatin, mevastatin , lovastatin or pharmaceutically acceptable salts thereof.

4. A pharmaceutical composition of claim 3 comprising amlodipine besylate.

5. A first pharmaceutical composition for use with a second pharmaceutical composition for achieving a antihypertensive effect and a hypolipidemic effect in a mammal suffering from hypertension and hyperlipidemia, which effects are greater than the sum of the antihypertensive and hypolipidemic effects achieved by administering said first and second pharmaceutical compositions separately and which second pharmaceutical composition comprises an amount of amlodipine or a pharmaceutically acceptable acid addition salt thereof and a pharmaceutically acceptable carrier or diluent, said first pharmaceutical composition comprising an amount of a statin or a pharmaceutically acceptable salt thereof and a pharmaceutically acceptable carrier or diluent; provided that said statin is not atorvastatin or a pharmaceutically acceptable salt thereof.

6. A composition of claim 5 wherein said statin is simvastatin, pravastatin, rivastatin, mevastatin, fluindostatin, velostatin, fluvastatin, dalvastatin, dihydrocompactin, compactin or lovastatin; or a pharmaceutically acceptable salt of simvastatin, pravastatin, rivastatin, mevastatin, fluindostatin, velostatin, fluvastatin, dalvastatin, dihydrocompactin, compactin or lovastatin.

7. A composition of claim 6 wherein said second pharmaceutical composition comprises amlodipine besylate.

8. A first pharmaceutical composition for use with a second pharmaceutical composition for achieving a antihypertensive effect and a hypolipidemic effect in a mammal suffering from hypertension and hyperlipidemia, which effects are greater than the sum of the antihypertensive and hypolipidemic effects achieved by administering said first and second pharmaceutical compositions separately and which second pharmaceutical composition comprises an amount of a statin or a pharmaceutically acceptable salt thereof and a pharmaceutically acceptable carrier or diluent, said first pharmaceutical composition comprising an amount of amlodipine or a pharmaceutically acceptable acid addition salt thereof and a pharmaceutically acceptable carrier or diluent, provided that said statin is not atorvastatin or a pharmaceutically acceptable salt thereof.

9. A composition of claim 8 wherein said statin is simvastatin, pravastatin, rivastatin, mevastatin, fluindostatin, velostatin, fluvastatin, dalvastatin, dihydrocompactin, compactin or lovastatin; or a pharmaceutically acceptable salt of simvastatin, pravastatin, rivastatin, mevastatin, fluindostatin, velostatin, fluvastatin, dalvastatin, dihydrocompactin, compactin or lovastatin.

10. A composition of claim 9 comprising amlodipine besylate.

11. A first pharmaceutical composition for use with a second pharmaceutical composition for achieving a antihypertensive effect and a hypolipidemic effect in a mammal suffering from hypertension and hyperlipidemia, which effects are greater than the antihypertensive and hypolipidemic effects achieved by administering said first or second pharmaceutical compositions separately and which second pharmaceutical composition comprises an amount of a statin or a pharmaceutically acceptable salt thereof and a pharmaceutically acceptable carrier or diluent, said first pharmaceutical composition comprising an amount of amlodipine or a pharmaceutically acceptable acid addition salt thereof and a pharmaceutically acceptable carrier or diluent; provided that said statin is not atorvastatin or a pharmaceutically acceptable salt thereof.

12. A composition of claim 11 comprising amlodipine besylate.

13. A first pharmaceutical composition for use with a second pharmaceutical composition for achieving a antihypertensive effect and a hypolipidemic effect in a mammal suffering from hypertension and hyperlipidemia, which effects are greater than the antihypertensive and hypolipidemic effects achieved by administering said first or second pharmaceutical compositions separately and which second pharmaceutical composition comprises an amount of amlodipine or a pharmaceutically acceptable acid addition salt thereof and a pharmaceutically acceptable carrier or diluent, said first pharmaceutical composition comprising an amount of a statin or a pharmaceutically acceptable salt thereof and a pharmaceutically acceptable carrier or diluent; provided that said statin is not atorvastatin or a pharmaceutically acceptable salt thereof.

14. A composition of claim 13 wherein said statin is simvastatin, pravastatin, rivastatin, mevastatin, fluindostatin, velostatin, fluvastatin, dalvastatin, dihydrocompactin, compactin or lovastatin; or a pharmaceutically acceptable salt of simvastatin, pravastatin, rivastatin, mevastatin, fluindostatin, velostatin, fluvastatin, dalvastatin, dihydrocompactin, compactin or lovastatin.

15. A first pharmaceutical composition for use with a second pharmaceutical composition for achieving an antianginal effect in a mammal suffering from angina pectoris, which effect is greater than the sum of the antiangina effects achieved by administering said first and second pharmaceutical compositions separately and which second pharmaceutical composition comprises an amount of a statin or a pharmaceutically acceptable salt thereof and a pharmaceutically acceptable carrier or diluent, said first pharmaceutical composition comprising an amount of amlodipine or a pharmaceutically acceptable acid addition salt thereof and a pharmaceutically acceptable carrier or diluent; provided that said statin is not atorvastatin or a pharmaceutically acceptable salt thereof.

16. A composition of claim 15 wherein said statin is simvastatin, pravastatin, rivastatin, mevastatin, fluindostatin, velostatin, fluvastatin, dalvastatin, dihydrocompactin, compactin or lovastatin; or a pharmaceutically acceptable salt of simvastatin, pravastatin, rivastatin, mevastatin, fluindostatin, velostatin, fluvastatin, dalvastatin, dihydrocompactin, compactin or lovastatin.

17. A composition of claim 16 comprising amlodipine besylate.

18. A first pharmaceutical composition for use with a second pharmaceutical composition for achieving an antianginal effect in a mammal suffering from angina pectoris, which effect is greater than the sum of the antiangina effects achieved by administering said first and second pharmaceutical compositions separately and which second pharmaceutical composition comprises an amount of amlodipine or a pharmaceutically acceptable acid addition salt thereof and a pharmaceutically acceptable carrier or diluent, said first pharmaceutical composition comprising an amount of a statin or a pharmaceutically acceptable salt thereof and a pharmaceutically acceptable carrier or diluent; provided that said statin is not atorvastatin or a pharmaceutically acceptable salt thereof.

19. A composition of claim 18 wherein said statin is simvastatin, pravastatin, -rivastatin, mevastatin, fluindostatin, velostatin,- fluvastatin, dalvastatin, dihydrocompactin, compactin or lovastatin; or a pharmaceutically acceptable salt of simvastatin, pravastatin, rivastatin, mevastatin, fluindostatin, velostatin, fluvastatin, dalvastatin, dihydrocompactin, compaction or lovastatin.

20. A composition of claim 19 wherein said second pharmaceutical composition comprises amlodipine besylate.

21. A first pharmaceutical composition for use with a second pharmaceutical composition for achieving an antianginal effect in a mammal suffering from angina pectoris, which effect is greater than the antianginal effects achieved by administering said first or second pharmaceutical compositions separately and which second pharmaceutical composition comprises an amount of a statin or a pharmaceutically acceptable salt thereof and a pharmaceutically acceptable carrier or diluent, said first pharmaceutical composition comprising an amount of amlodipine or-a pharmaceutically acceptable acid addition salt thereof and a pharmaceutically acceptable carrier or diluent; provided that said statin is not atorvastatin or a pharmaceutically acceptable salt thereof.

22. A composition of claim 21 comprising amlodipine besylate.

23. A first pharmaceutical composition for use with a second pharmaceutical composition for achieving an antianginal effect in a mammal suffering from angina pectoris, which effect is greater than the antianginal effects achieved by administering said first or second pharmaceutical compositions separately and which second pharmaceutical composition comprises an amount of amlodipine or a pharmaceutically acceptable acid addition salt thereof and a pharmaceutically acceptable carrier or diluent, said first pharmaceutical composition comprising an amount of .a statin or a pharmaceutically acceptable salt thereof and a pharmaceutically acceptable carrier or diluent; provided that said stain is not atorvastatin or a pharmaceutically acceptable salt thereof.

24. A composition of claim 23 wherein said stain is simvastatin, pravastatin, rivastatin, mevastatin, fluindostatin, velostatin, fluvastatin, dalvastatin, dihydrocompactin, compactin or lovastatin; or a pharmaceutically acceptable salt of simvastatin, pravastatin, rivastatin, mevastatin, fluindostatin velostatin, fluvastatin, dalvastatin, dihydrocompactin, compactin or lovastatin.

25. A first pharmaceutical composition for use with a second pharmaceutical composition for achieving an antiatherosclerotic effect in a mammal, which effect is greater than the sum of the antiatherosclerotic effects achieved by administering said first and second pharmaceutical compositions separately and which second pharmaceutical composition comprises an amount of amlodipine or a pharmaceutically acceptable acid addition salt thereof and a pharmaceutically acceptable carrier or diluent, said first pharmaceutical composition comprising an amount of a statin or a pharmaceutically acceptable salt thereof and a pharmaceutically acceptable carrier or diluent, provided that said statin is not atorvastatin or a pharmaceutically acceptable salt thereof.

26. A composition of claim 25 wherein said statin is simvastatin, pravastatin, rivastatin, mevastatin, fluindostatin, velostatin, fluvastatin, dalvastatin, dihydrocompactin, compactin or lovastatin; or a pharmaceutically acceptable salt of simvastatin, pravastatin, rivastatin, mevastatin, fluindostatin, velostatin, fluvastatin dalvastatin, dihydrocompactin, compactin or lovastatin.

27. A composition of claim 26 wherein said second pharmaceutical composition comprises amlodipine besylate.

28. A composition of claim 27 wherein said antiatherosclerotic effect is manifested by a slowing of the progression of atherosclerotic plaques.

29. A composition of claim 28 wherein said progression of atherosclerotic plaques is slowed in coronary arteries.

30. A composition of claim 28 wherein said progression of atherosclerotic plaques is slowed in carotid arteries.

31. A composition of claim 28 wherein said progression of atherosclerotic plaques is slowed in the peripheral arterial system.

32. A composition of claim 27 wherein said antiatherosclerotic effect is manifested by a regression of atherosclerotic plaques.

33. A composition of claim 32 wherein said regression of atherosclerotic plaques occurs in coronary arteries.

34. A composition of claim 32 wherein said regression of atherosclerotic plaques occurs in carotid arteries.

35. A composition of claim 32 wherein said regression of atherosclerotic plaques occurs in the peripheral arterial system.

36. A first pharmaceutical composition for use with a second pharmaceutical composition for achieving an antiatherosclerotic effect in a mammal, which effect is greater than the sum of the antiatherosclerotic effects achieved by administering said first and second pharmaceutical compositions separately and which second pharmaceutical composition comprises an amount of a statin or a .pharmaceutically acceptable salt thereof and a pharmaceutically acceptable carrier or diluent, said first pharmaceutical composition comprising an amount of amlodipine or a pharmaceutically acceptable acid addition salt thereof and a pharmaceutically acceptable carrier or diluent; provided that said statin is not atorvastatin or a pharmaceutically acceptable salt thereof.

37. A composition of claim 36 wherein said statin is simvastatin, pravastatin, rivastatin, mevastatin, fluindostatin, velostatin, fluvastatin, dalvastatin, dihydrocompactin, compactin or lovastatin; or a pharmaceutically acceptable salt of simvastatin, pravastatin, rivastatin,- mevastatin, fluindostatin, velostatin, fluvastatin, dalvastatin, dihydrocompactin, compactin or lovastatin.

38. A composition of claim 37 comprising amlodipine besylate.

39. A composition of claim 38 wherein said antiatherosclerotic effect is manifested by a slowing of the progression of atherosclerotic plaques.

40. A composition of claim 39 wherein said progression of atherosclerotic plaques is slowed in coronary arteries.

41. A composition of claim 39 wherein said progression of atherosclerotic plaques is slowed in carotid arteries.

42. A composition of claim 39 wherein said progression of atherosclerotic plaques is slowed in the peripheral arterial system.

43. A composition of claim 38 wherein said antiatherosclerotic effect: is manifested by a regression of atherosclerotic plaques.

44. A composition of claim 43 wherein said regression of atherosclerotic plaques occurs in coronary arteries.

45. A composition of claim 43 wherein said regression of atherosclerotic plaques occurs in carotid arteries.

46. A composition of claim 43 wherein said regression of atherosclerotic plaques occurs in the peripheral arterial system.

47. A first pharmaceutical composition for use with a second pharmaceutical composition for achieving an antiatherosclerotic effect in a mammal, which effect is greater than the antiatherosclerotic effects achieved by administering said first or second pharmaceutical compositions separately and which second pharmaceutical composition comprises an amount of a statin or a pharmaceutically acceptable salt thereof and a pharmaceutically acceptable carrier or diluent, said first pharmaceutical composition comprising an amount of amlodipine or a pharmaceutically acceptable acid addition salt thereof and a pharmaceutically acceptable carrier or diluent provided that said statin is not atorvastatin or a pharmaceutically acceptable salt thereof.

48. A composition of claim 47 comprising amlodipine besylate.

49. A first pharmaceutical composition for use with a second pharmaceutical composition for achieving an antiatherosclerotic effect in a mammal, which effect is greater than the antiatherosclerotic effects, achieved by administering said first or second pharmaceutical compositions separately and which second pharmaceutical composition comprises an amount of amlodipine or a pharmaceutically acceptable acid addition salt thereof and a pharmaceutically acceptable carrier or diluent, said first pharmaceutical composition comprising an amount of a statin or a pharmaceutically acceptable salt thereof and a pharmaceutically acceptable carrier or diluent; provided that said statin is not atorvastatin or a pharmaceutically acceptable salt thereof.

50. A composition of claim 49 wherein said statin is simvastatin, pravastatin, rivastatin, mevastatin, fluindostatin, velostatin, fluvastatin, dalvastatin, dihydrocompactin, compactin or lovastatin; or a pharmaceutically acceptable salt of simvastatin, pravastatin, rivastatin, mevastatin, fluindostatin, velostatin, fluvastatin, dalvastatin, dihydrocompactn, compactin or lovastatin.

51. A first pharmaceutical composition for use with a second pharmaceutical composition for managing cardiac risk in a mammal at risk of suffering an adverse cardiac event, which effect is greater than the sum of the cardiac risk management effects achieved by administering said first and second pharmaceutical compositions separately and which second pharmaceutical composition comprises an amount of a statin or a pharmaceutically acceptable salt thereof and a pharmaceutically acceptable carrier or diluent, said first pharmaceutical composition comprising an amount of amlodipine or a pharmaceutically acceptable acid addition salt thereof and a pharmaceutically acceptable carrier or diluent, provided that said statin. Is not atorvastatin or a pharmaceutically acceptable salt thereof.

52. A composition of claim 51 wherein said statin is simvastatin, pravastatin, rivastatin, mevastatin, fluindostatin, velostatin, fluvastatin, dalvastatin, dihydrocompactin, compactin or lovastatin; or a pharmaceutically acceptable salt of simvastatin, pravastatin, rivastatin, mevastatin, fluindostatin, velostatin, fluvastatin, dalvastatin, dihydrocompactin, compactin or lovastatin.

53. A composition of claim 52 comprising amlodipine besylate.

54. A first pharmaceutical composition for use with a second pharmaceutical composition for managing cardiac risk in a mammal at risk of suffering.;an adverse cardiac event, which effect is greater than the sum of the cardiac risk management effects achieved by administering said first and second pharmaceutical compositions separately and which second pharmaceutical composition comprises an amount of amlodipine or a pharmaceutically acceptable acid addition salt thereof and a pharmaceutically acceptable carrier or diluent, said first pharmaceutical composition comprising an amount of a statin or a pharmaceutically acceptable salt thereof and a pharmaceutically acceptable carrier or diluent, provided that said statin is not atorvastatin or a pharmaceutically acceptable salt thereof.

55. A composition of claim 54 wherein said statin is simvastatin, pravastatin, rivastatin, mevastatin, fluindostatin, velostatin, fluvastatin, dalvastatin, dihydrocompactin, compactin or lovastatin; or a pharmaceutically acceptable salt of simvastatin, pravastatin, rivastatin, mevastatin, fluindostatin, velostatin, fluvastatin, dalvastatin, dihydrocompactin, compactin or lovastatin.

56. A composition of claim 55 wherein said second pharmaceutical composition comprises amlodipine besylate.

57. A first pharmaceutical composition for use with a second pharmaceutical composition for managing cardiac risk in a mammal at risk of suffering an adverse cardiac event, which effect is greater than the cardiac risk management effects achieved by administering said first or second pharmaceutical compositions separately and which second pharmaceutical composition comprises an amount of a statin or a pharmaceutically acceptable salt thereof and a pharmaceutically acceptable carrier or diluent, said first pharmaceutical composition comprising an amount of amlodipine or a pharmaceutically acceptable acid addition salt thereof and a pharmaceutically acceptable carrier or diluent; provided that said statin is not atorvastatin or a pharmaceutically acceptable salt thereof.

58. A composition of claim 57 comprising amlodipine besylate.

59. A first pharmaceutical, composition for use with a second pharmaceutical composition for managing cardiac risk in a mammal at risk of suffering an adverse cardiac event, which effect is greater than the cardiac risk management effects achieved by administering said first or second pharmaceutical compositions separately and which second pharmaceutical composition comprises an amount of amlodipine or a pharmaceutically acceptable acid addition salt thereof and a pharmaceutically acceptable carrier or diluent, said first pharmaceutical composition comprising an amount of a statin or a pharmaceutically acceptable salt thereof and a pharmaceutically acceptable carrier or diluent; provided that said statin is not atorvastatin or a pharmaceutically acceptable salt thereof.

60. A composition of claim 59 wherein said statin is simvastatin, pravastatin, rivastatin, mevastatin, fluindostatin, velostatin, fluvastatin, dalvastatin, dihydrocompactin., compactin or lovastatin; or a pharmaceutically acceptable salt of simvastatin, pravastatin, rivastatin, mevastatin, fluindostatin, velostatin, fluvastatin, dalvastatin, dihydrocompactin, compactin or lovastatin.

61. A kit for achieving a therapeutic effect in a mammal comprising:
a. an amount of amlodipine or a pharmaceutically acceptable acid addition salt thereof and a pharmaceutically acceptable carrier or diluent in a first unit dosage form;
b. an amount of a statin or a pharmaceutically acceptable salt thereof and a pharmaceutically acceptable carrier or diluent in a second unit dosage form; and
c. container means for containing said first and second dosage forms; provided that said statin is not atorvastatin or a pharmaceutically acceptable salt thereof.

62. A kit of claim 61 wherein said statin is simvastatin, pravastatin, dalvastatin, mevastatin, fluindostatin, velostatin, fluvastatin, dalvastatin, dihydrocompactin, compactin or lovastatin; or a pharmaceutically acceptable salt of simvastatin, pravastatin, rivastatin, mevastatin, fluindostatin, velostatin, fluvastatin, dalvastatin, dihydrocompactn, compactin or lovastatin.

63. A kit of claim 62 comprising amlodipine besylate.

64. A kit of claim 61 wherein said therapeutic effect is treatment of hypertension and hyperlipidemia.

65. A kit of claim 61 wherein said therapeutic effect is treatment of angina pectoris.

66. A kit of claim 61 wherein said therapeutic effect is management of cardiac risk

67. A kit of claim 61 wherein said therapeutic effect is treatment of atherosclerosis.

68. A kit of claim 67 wherein said treatment of atherosclerosis slows the progression of atherosclerotic plaques.

69. A kit of claim 68 wherein said progression of atherosclerotic plaques is slowed in coronary arteries.

70. A kit of claim 68 wherein said progression of atherosclerotic plaques is slowed in carotid arteries.

71. A kit of claim 68 wherein said progression of atherosclerotic plaques is slowed in the peripheral arterial system.

72. A kit of claim 67 wherein said treatment of atherosclerosis causes the regression of atherosclerotic plaques.

73. A kit of claim 72 wherein said regression of atherosclerotic plaques occurs in coronary arteries.

74. A kit of claim 72 wherein said regression of atherosclerotic plaques occurs in carotid arteries.

75. A kit of claim 72 wherein said regression of atherosclerotic plaques occurs in the peripheral arterial system.

76. A kit of claim 63 wherein said therapeutic effect is treatment of hypertension and hype dipidemia.

77. A kit of claim 63 wherein said therapeutic effect is treatment of angina pectoris.

78. A kit of claim 63 wherein said therapeutic effect is treatment of cardiac risk.

79. A kit of claim 63 wherein said therapeutic effect is treatment of atherosclerosis.

80. A kit of claim 79 wherein said treatment of atherosclerosis slows the progression of atherosclerotic plaques.

81. A kit of claim 80 wherein said progression of atherosclerotic plaques is slowed in coronary arteries.

82. A kit of claim 80 wherein said progression of atherosclerotic plaques is slowed in carotid arteries.

83. A kit of claim 80 wherein said progression of atherosclerotic plaques is slowed in the peripheral arterial system.

84. A kit of claim 79 wherein said treatment of atherosclerosis causes the regression of atheroscleroticplaques.

85. A kit of claim 84 wherein said regression of atherosclerotic plaques occurs in coronary arteries.

86. A kit of claim 84 wherein said regression of atherosclerotic plaques occurs in carotid arteries.

87. A kit of claim 84 wherein said regression of atherosclerotic plaques occurs in the peripheral arterial system.

88. A method for treating a mammal in need of therapeutic treatment comprising administering to said mammal
(a) an amount of a first compound, said first compound being amlodipine or a pharmaceutically acceptable acid addition salt thereof; and
(b) an amount of a second compound, said second compound being statin or a pharmaceutically acceptable salt thereof;
wherein said first compound and said second compound are each optionally and independently administered together with a pharmaceutically acceptable carrier or diluent; provided that said statin is not atorvastatin or a pharmaceutically acceptable salt thereof.

89. A method of claim 88 wherein said stain is simvastatin, pravastatin, rivastatin, mevastatin, fluindostatin, velostatin, fluvastatin, dalvastatin, dihydrocompactin, compactin or lovastatin; or a pharmaceutically acceptable salt-of simvastatin, pravastatin, rivastatin, mevastatin, fluindostatin, velostatin, fluvastatin, dalvastatin, dihydrocompactin, compactin or lovastatin.

90. A method of claim 89 comprising amlodipine besylate.

91. A method of claim 88 wherein said first compound and said second compound are administered simultaneously.

92. A method of claim 88 wherein said first compound and said second compound are administered sequentially in either order.

93. A method of claim 90 wherein said first compound and said second compound are administered simultaneously.

94. A method of claim 90 wherein said first compound and said second compound are administered sequentially in either order.

95. A method of claim 88 wherein said therapeutic treatment comprises antihypertensive treatment and antihyperlipidemic treatment

96. A method of claim 93 wherein said therapeutic treatment comprises antihypertensive treatment and aniihyperlipidemic treatment

97. A method of claim 94 wherein said therapeutic treatment comprises antihypertensive treatment and antihyperlipidemic treatment

98. A method of claim 88 wherein said therapeutic treatment comprises antianginal treatment.

99. A method of claim 93 wherein said therapeutic treatment comprises antianginal treatment

100. A method of claim 94 wherein said therapeutic treatment comprises antianginal treatment.

101. A method of claim 88 wherein said therapeutic treatment comprises cardiac risk management

102. A method of claim 93 wherein said therapeutic treatment compnrises cardiac risk management

103. A method of claim 94 wherein said therapeutic treatment comprises cardiac risk management

104. A method of claim 88 wherein said therapeutic treatment comprises antiatherosclerotic treatment

105. A method of claim 93 wherein said therapeutic treatment comprises antiatherosclerotic treatment

106. A method of claim 94 wherein said therapeutic treatment comprises antiatherosclerotic treatment
